(19) United States
(12) Patent Application Publication
(10) Pub. No.: US 2004/0063123 A1
(43) Pub. Date: Apr. 1, 2004

(54) Method of collecting data for estimation of susceptibility to periodontal disease

(76) Inventors: Yoshihiro Abiko, Sapporo-shi (JP); Tohru Kaku, Sapporo-shi (JP); Toshiya Arakawa, Sapporo-shi (JP); Taishin Takuma, Sapporo-shi (JP); Michiko Nishimura, Ebetsu-shi (JP); Kaoru Kusano, Sapporo-shi (JP)

Correspondence Address: Leonard W. Sherman, Sherman & Shalloway, 413 N. Washington Street, Alexandria, VA, 22314, US

(21) Appl. No.: 10/629,613

(22) Filed: Jul. 30, 2003

Related U.S. Application Data
(63) Continuation-in-part of application No. 10/300,799, filed on Nov. 21, 2002.

(30) Foreign Application Priority Data
Mar. 5, 2002 (JP) 2002-058955

(51) Int. Cl. C12Q 1/68; A61C 5/00

(52) U.S. Cl. 435/6; 433/217.1

(57) ABSTRACT

A method is provided, which comprises examining the regulating activity of a defensin gene promoter closely associated with the expression activity of defensin, an antibacterial peptide, on the basis of the detection of a gene mutation, and estimating future susceptibility to periodontal disease. The sequence homology between DNA derived from a defensin gene promoter existing in a sample obtained from the gingival tissues of a subject and a nucleotide sequence comprising a mutation site in the promoter region, and/or compatibility in PCR amplification, are determined. Thereby a gene mutation is detected and then the site of the gene mutation is determined, so that data for the estimation can be collected.

### CROSS-REFERENCES TO RELATED APPLICATIONS

This is a continuation-in-part application of application Ser. 10/300,799, filed Nov. 21, 2002.

### TECHNICAL FIELD

The present invention relates to a method of collecting data for estimating susceptibility to periodontal disease and nucleotide sequences used in the data collection. Specifically, the present invention relates to nucleotide sequences comprising mutant sequence portions in a defensin gene promoter region that regulates the expression of defensin, an antibacterial peptide; and a method of collecting data for estimating susceptibility to periodontal disease wherein the variations of the promoter activity of regulating the expression activity of the defensin gene are examined using the above nucleotide sequences so as to obtain the above data.

### BACKGROUND ART

Periodontal disease is a chronic inflammatory disease of periodontal tissues caused by dental plaque bacteria. A number of periodontal disease-related bacteria have been specified, which are directly or indirectly associated with that disease. When periodontitis is developed, factors associated with the innate immunity of gingival epithelial cells exerting a protective function such as cytokines, adhesive factors and antibacterial substances, intervene in the activity of the periodontal disease-related bacteria. Defensin is a microbicidal peptide known as such an antimicrobial substance.

Defensins are peptides having an antimicrobial activity against Gram-positive bacteria, Gram-negative bacteria, fungi and envelope viruses, and these peptides are classified into α type and β type. Examples of α type defensins, which have been reported, include a total 6 types such as HNP-1 to -4 localized in azurophilic granules of human neutrophils and human defensin-5 and -6 localized in Paneth cells in human small intestine. Examples of β type so far found include LAP (lingual antimicrobial peptide) and TAP (trancheal anti-microbial peptide), which are localized in bovine tongue and trachea, and both of them act as epithelium-derived protective factors against infection.

Recently, techniques of predicting susceptibility to a certain disease by examining a specific substance existing in vivo have been proposed. Many of the techniques, however, detect a disease-causing or disease-associated substance or detect a disease-related gene by a certain method. Such techniques will not be established as an effective method of predicting future susceptibility to the diseases, if the relationship between a target disease and a causative gene, and a mechanism of controlling expression of the gene are not clarified in a molecular level.

One of diseases which are under such circumstances is the above described periodontal disease. Some methods of estimating the genes of the disease-associated bacteria have been established, but these methods can solely specify the disease-associated bacteria. As is the case with other diseases, a technique of evaluating the possibility of being affected by periodontal disease in future by analysis of genes closely associated with the development of this disease is awaited.

Studies of genes associated with periodontal disease have been progressing so far. Both for α and β defensins their genes exist on chromosome 8, and their expression is regulated.

To date, some of reports, for example, indicate that severe periodontitis is caused by the mutation of a LFA-1 gene (e.g. refer to nonpatent publication 1) or the mutation of a cathepsin C gene (e.g. refer to nonpatent publication 2).

Another report indicates a possibility that genes for such as FcγRIIIa (CD16) (e.g. refer to nonpatent publication 3), HLA-DR and HLA-DQ (e.g. refer to nonpatent publication 4), IL-1 (e.g. refer to nonpatent publication 5), TNF-β an angiotensin converting enzyme, endothelin (e.g. refer to nonpatent publication 6) and the like are associated with periodontal disease. However, with these findings alone, it is still insufficient to establish a method predicting the onset of a periodontal disease.

Moreover, with regard to human β-defensin, there are no methods of confirming the presence or absence of the expression of β-defensin or its expression level by determining a specific nucleotide sequence of DNA in the gene, thereby estimating a risk or susceptibility to diseases caused by bacteria such as periodontal disease.

Nonpatent Publication 1:

Springer T A, Thompson W S et al.: Inherited deficiency of the Mac-1, LFA-1, p.150, 95 glycoprotein family and its molecular basis. “J. Exp. Med”, 1984, Vol. 160, p. 1901-1908

Nonpatent Publication 2:

Toomes C, James J et al.: Loss of function mutations in the cathepsin C gene results in periodontal disease and palmoplantar keratosis, “Nature Genetics”, 1999, Vol. 23, p. 421-424

Nonpatent Publication 3:

Kobayashi T, Westerdaal N A et al.: Relevance of immunoglobulin G Fc receptor polymorphism to recurrence of adult periodontitis in Japanese patients, “Infect. Immun.”, 1997, Vol. 65, p. 3556-3560

Nonpatent Publication 4:

Takashiba S, Ohyama H et al.: HLA genetics for diagnosis of susceptibility to early-onset periodontitis, “J. Periodont. Res.”, 1999, Vol. 34, p. 374-378

Nonpatent Publication 5:

Kornman K S, Crane A et al.: The interleukin-1 genotype as a severity factor in adult periodontal disease, “J. Clin. Periodontol.”, 1997, Vol. 24, p. 72-77

Nonpatent Publication 6:

Holla L I, Fassmann A et al.: Interactions of lymphotoxin alpha (TNF-beta), angiotensin-converting enzyme (ACE), and endothelin-1 (ET-1) gene polymorphisms in adult periodontitis, “J. Periodontol.”, 2001, Vol. 72, p. 85-89

### DISCLOSURE OF THE INVENTION

The present inventors have focused attention on fluctuations in the amount of generated defensin, an antimicrobial peptide, which are closely associated with the onset of periodontal disease, and have conducted studies regarding the nucleotide sequence of a gene causing the expression of defensin. As a result, the present inventors have found that, among defensin genes from an established cell line, a mutant base is present in the nucleotide sequence of the gene regulating the expression of defensin, and they have succeeded to determine the mutation site. On the basis of these findings, the present inventors have accomplished the present invention, which relates to a method of estimating susceptibility to periodontal disease.

It is an object of the present invention to provide a new method of testing and collecting data for evaluation so as to inform patients and/or dentists of susceptibility to periodontal disease caused by decrease of an antibacterial ability.

Further, it is another object of the present invention to provide reagents (probes and primers), test materials (a kit and a DNA chip) and others, which are required to carry out the above method.

The summary of the present invention is as follows.

The method of the present invention is a method of collecting data for estimating susceptibility to periodontal disease wherein the method comprises: in order to detect the presence of a gene mutation and/or a mutation site existing in the promoter region of a human defensin gene in a sample, by using a nucleotide sequence being a part of the promoter of the defensin gene and comprising a mutant base as a nucleotide sequence for a probe, determining

(i) a hybridization site of hybridization between the defensin gene promoter nucleotide sequence in the sample and said probe, and/or

(ii) an amplification ability in gene amplification where primers comprising said probe are used; thereby clarifying the change of the activity of the defensin promoter to regulate the expression of the defensin gene based on the thus detected presence of a gene mutation and/or a mutation site.

In a preferred embodiment of the above described method of the present invention, the above method comprises: in order to detect the presence of a gene mutation and/or a mutation site existing in the promoter region of a human β-defensin 2 gene in a sample,

by using a nucleotide sequence being a part of the promoter of the β-defensin 2 gene and comprising a mutant base as a nucleotide sequence for a probe,

determining

(i) a hybridization site of hybridization between the β-defensin 2 gene promoter nucleotide sequence in the sample and said probe, and/or

(ii) an amplification ability in gene amplification where primers comprising said probe are used; thereby clarifying the change of the activity of the human β-defensin 2 promoter to regulate the expression of the β-defensin 2 gene based on the thus detected presence of a gene mutation and/or a mutation site.

The nucleotide sequence of the present invention used to obtain data for estimating susceptibility to periodontal disease is used to detect a mutant type sequence existing in the promoter region of the human β-defensin 2 gene, and comprises sequences of at least 5 nucleotides being each of upstream and downstream from a mutation site, or otherwise at-least-10-nucleotide-containing sequences of which 3′ terminus is the nucleotide of a mutation site in the promoter nucleotide sequences.

The above nucleotide sequence is a nucleotide sequence used as a probe, in which the above nucleotide sequence is preferably any sequence selected from: a DNA nucleotide sequence amplified by primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

wherein G is substituted by C at a site −1431 (mutation site 1) located upstream of the transcription initiation point of the human β-defensin 2 gene, and/or

a DNA nucleotide sequence amplified by primer set 2:

| | |
|---|---|
| 5′ TCTTTCTCAAACTGCCCTTAG 3′ | (SEQ ID NQ:3) |
| 5′ ATGGGATTGTGACTACATGTG 3′ | (SEQ ID NO:4) |

wherein G is substituted by T at a site −1035 (mutation site 2-1), and/or A is substituted by G at a site −1027 (mutation site 2-2), and/or G is substituted by A at a site-936 (mutation site 2-3), and/or C is substituted by T at a site-923 (mutation site 2-4),

and/or a DNA nucleotide sequence amplified by the same primer set as above, wherein T is substituted by C at a site −912 (mutation site 2-5), and/or G is substituted by A at a site −874 (mutation site 2-6), and/or

a DNA nucleotide sequence amplified by primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

wherein C is substituted by T at a site −539 (mutation site 3-1), and/or A is substituted by G at a site −472 (mutation site 3-2), and/or

a DNA nucleotide sequence amplified by primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

wherein T is substituted by C at a site −108 (mutation site 4).

Moreover, the above nucleotide sequence may be further modified with markers for detection and/or amplification.

The primer of the present invention comprises both nucleotide sequences of primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

and is used to amplify DNA derived from the human defensin gene.

Moreover, the primer of the present invention comprises both nucleotide sequences of primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

and is used to amplify DNA derived from the human defensin gene.

Furthermore, the primer of the present invention comprises both nucleotide sequences of primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

and is used to amplify DNA derived from the human defensin gene.

Still further, the primer of the present invention comprises both nucleotide sequences of primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

and is used to amplify DNA derived from the human defensin gene.

Further, the primer of the present invention also includes primers comprising any one of the above described probes and being used to determine an amplification ability in gene amplification.

The kit of the present invention is a kit used to estimate susceptibility to periodontal disease, and it comprises at least one type of probe comprising a nucleotide sequence used as a probe selected from the above described nucleotide sequences for probes and further comprises any of the above described primers as necessary, so as to detect a mutation site existing in the promoter region of the human defensin gene.

The DNA chip of the present invention comprises at least one type of nucleotide sequence used as a probe selected from at least the above described nucleotide sequences for probes and further comprises any of the above described primers as necessary.

The prediction method of the susceptibility of periodontal diseases according to the present invention comprises an analysis of human alleles using the allele-specific PCR method and estimation of the susceptibility of periodontal disease on the basis of the elucidated nucleotide sequences.

The above described method of the present invention is a method wherein hybridization is carried out under stringent conditions.

The data processing system of the present invention is a system wherein it captures a detection signal generated from a detecting device associated with the above described kit or DNA chip and examines the activity of the human β-defensin 2 promoter to regulate the expression of the β-defensin 2 gene, so as to provide data for estimating susceptibility to periodontal disease.

### BRIEF DESCRIPTION OF THE DRAWINGS

FIG. 1A shows a part of the nucleotide sequence (1,020 bases) of the human β-defensin 2 gene.

FIG. 1B is a continuation of FIG. 1A and shows a part of the nucleotide sequence (1,020 bases) of the human β-defensin 2 gene.

FIG. 1C is a continuation of FIG. 1B and shows a part of the nucleotide sequence (1,020 bases) of the human β-defensin 2 gene.

FIG. 1D is a continuation of FIG. 1C and shows a part of the nucleotide sequence (1,020 bases) of the human β-defensin 2 gene.

FIG. 1E is a continuation of FIG. 1D and shows a part of the nucleotide sequence (719 bases) of the human β-defensin 2 gene.

All the nucleotide sequence of the human β-defensin 2 gene comprise a promoter region as well as a coding region which starts from position 2732.

The DNA sequences of human β-defensin 2 gene are described in the following publication:

Diamond, G., Kaiser, V, Rhodes, J., Russell, J. P., and Bevins, C. L. Transcriptional regulation of beta-defensin gene expression in tracheal epithelial cells. Infect. Immun. 68(1), p113-119 (2000)

### DETAILED DESCRIPTION OF THE INVENTION

The present invention will be explained below with regard to the expression of a defensin gene, a promoter regulating the expression, the detection of a gene mutation in the promoter region, a probe comprising a mutant base, a method of collecting data for estimating susceptibility to periodontal disease by the use of the probe, and a method of processing the obtained data.

The term “nucleic acid” as used herein means a polynucleotide including DNA and RNA in the present specification, and the term “nucleotide sequence” is herein used to designate the nucleotide sequence of nucleic acid. The term, “base sequence” is also used to refer to it.

Moreover, the term “mutation” means that the original base of defensin gene nucleotides is substituted by another type of base, for example, by point mutation, and the thus substituted base is referred to as a “mutant base.” Another types of bases include a base modified by methylation or the like. Herein, “gene mutation” also is used to mean a mutation occurred at nucleotide bases of gene.

A base position in the gene is herein specified according to the conventional practice of setting the translation initiation point (atg) of the human defensin gene as a starting point; a position upstream thereof is expressed as a minus number and a position downstream thereof is expressed as a plus number. Further, the term “periodontal disease” is used as a generic name embracing gingivitis, periodontitis, alveolar pyorrhea, and others.

(A) Expression of Human Defensin and Its Regulation

The feature common to α-defensin and β-defensin is that both defensins have a wide range of antimicrobial spectrum against bacteria, fungi, a virus or the like (Ganz T: defensins and host defense, Science 1999; 286; 420-421).

From the observation of gingival crevicular fluid (GCF) present in gingival crevices, it is suggested that the gingiva is protected by both α-defensin and β-defensin. This is completely the same as the observation of the bowel in which the crypts are protected by α-defensin and the epithelium covering the intestinal villi expresses β-defensin.

Expression of β-Defensin in the Oral Cavity

The β-defensin is expressed in the gingiva, the tongue, the salivary gland and other oral tissues. Three types of β-defensins (hBD-1, hBD-2 and hBD-3) are expressed in human oral epithelium and oral keratinocytes. The human β-defensin 1 (hBD-1) is expressed in many epithelial tissues including the kidney, the gastrointestinal tract, the respiratory apparatus, the stratified epithelium in the oral cavity and others. The expression of hBD-1 in these organs is induced by inflammation, lipopolysaccharide from Gram-negative bacteria and pro-inflammatory cytokines. The human β-defensin 2 (hBD-2) and the human β-defensin 3 (hBD-3) have been isolated from the skin of a patient with psoriasis, and it has been shown that a larger amount of hBD-2 and hDB-3 was secreted therein than in normal skin. The hBD-2 was also expressed in the oral epithelium, and the expression was induced depending on inflammation. Preliminary data suggests that the hBD-3 also is expressed in oral keratinocytes.

In normal gingival tissues, the above described antibacterial peptide is detected in an upper spinous cell layer, a granule cell layer and a horny layer, whereas mRNA of both hBD-1 and hBD-2 is strongly expressed particularly in the spinous cell layer. The strongest expression is observed in a region where plaque is formed on the surface of dentin, or a gingival margin contacting with the gingival crevice in an inflammatory state. These positions correspond to sites where the above peptide plays its role as an antibacterial barrier of the epithelium. However, hBD-1 and hBD-2 are not detected in the junctional epithelium where the cells are relatively undifferentiated. Accordingly, the findings on the lack of the expression of β-defensins in the junctional epithelium, the localization of β-defensins in the basal layer of the stratified epithelium, and differentiation in in vitro experiments, all show that the expression of β-defensin in the stratified epithelium depends on normal differentiation.

The normal gingiva with no inflammation expresses both hBD-1 and hBD-2 together. Substantially, the β-defensin, which exists as a product or is inducible, is detected in all the gingival biopsy samples, which are taken under either the normal non-inflammatory or inflammatory conditions. These mean that, in contrast to the normal epidermis, trachea and gastrointestinal tract, the normal non-inflammatory oral epithelium is activated and expresses hBD-2. The accentuation of hBD-2 seems to be a part of the normal barrier activity of the oral epithelium, which does not accompany the accentuation of a marker such as IL-8 for the intrinsic immune response of a host.

Regulation of β-Defensin Expression in the Oral Cavity

In view of much evidence obtained so far, it is suggested that the secretion of human β-defensin 2(hBD-2) is promoted by bacteria and inflammatory stimuli, and it is assumed that signals are transmitted via an NF-kB transcription factor. Moreover, the hBD-2 promoter region has 3 types of NF-kB binding sequences. (Liu L, Zhao C, Heng H H A, Ganz T,: The human beta defensin-1 and alpha defensin are encoded by adjacent genes; two families with differing disulfide topology share a common ancestry. Genomics 1997; 43; 316-320.) (Liu L, Wang L, Jia H P, et al.: Structure and mapping of the human beta-defensin gene and its expression at sites of inflammation, Gene 1998; 222; 237-244.) These signaling pathways are important for cells to respond to the inflammatory stimuli (Kopp E B, Ghosh S,: NF-kB and Rel proteins in innate immunity, Adv Immunol 1995; 58; 1-27). However, the regulation of transcription of hBD-2 cannot be substantially explained by only such a single regulation via NF-kB.

Using an RT-PCR (Reverse-Transcriptase Polymerization Chain Reaction) assay, the present inventors have shown so far that oral epithelial cells other than KB cells express hBD-2 mRNA (Cancer Lett. 143: 37-43, 1999) Because of its susceptibility to oral bacteria, the KB cell strain is often used for in vitro model of periodontitis. Thus, the present inventors, as a part of the studies of the present invention, have carried out the following experiment to search for transcription factors and transcription regulating sequence elements, responsible for the depletion of the expression of hBD-2 mRNA in the KB cells.

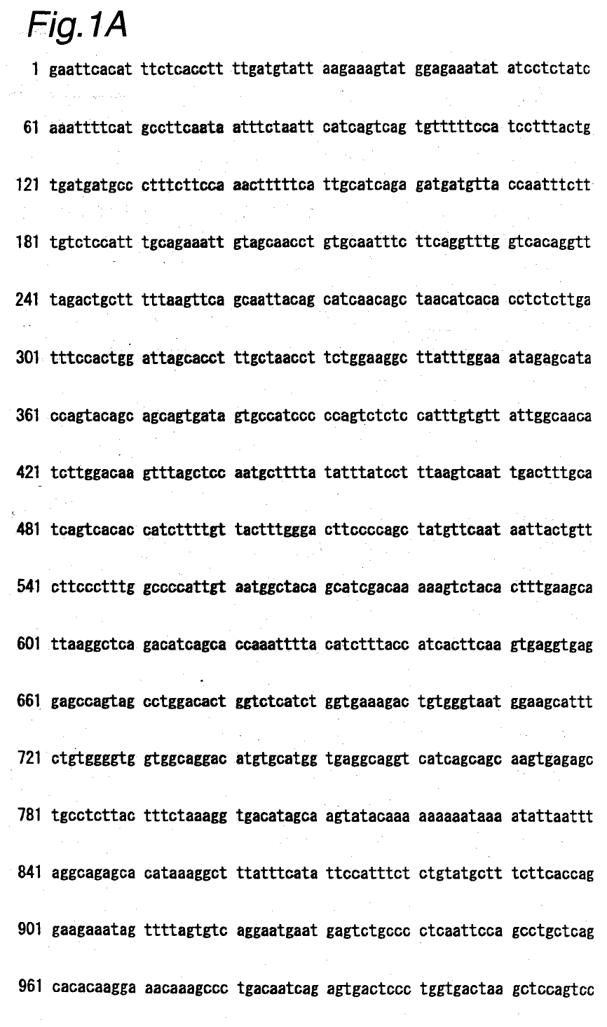

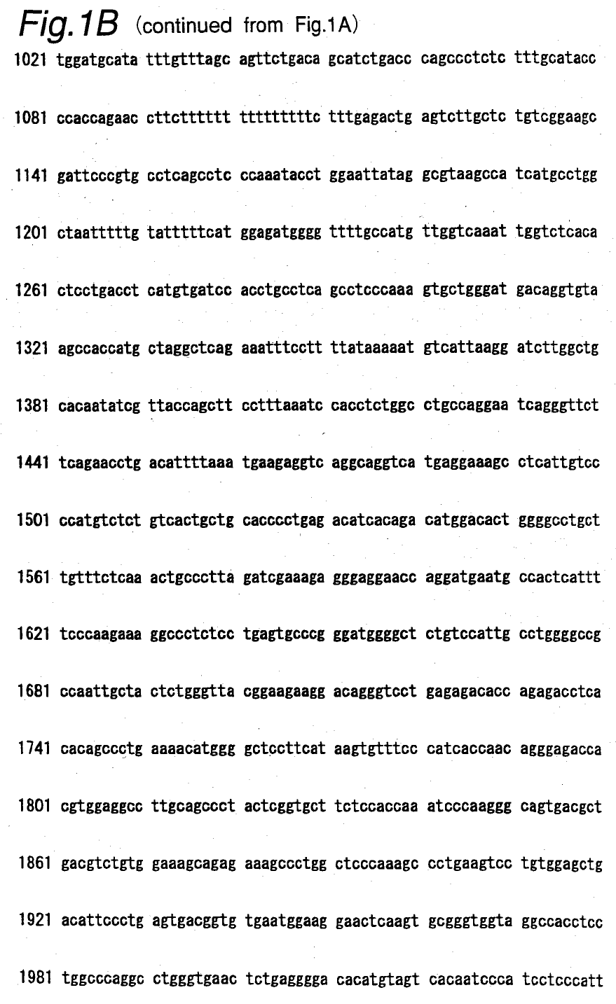

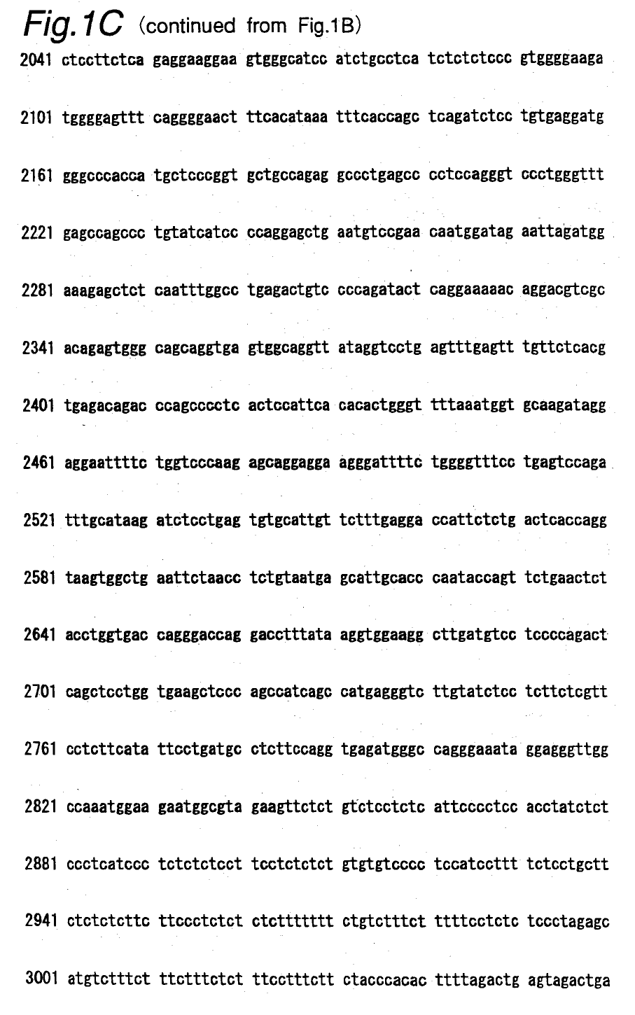

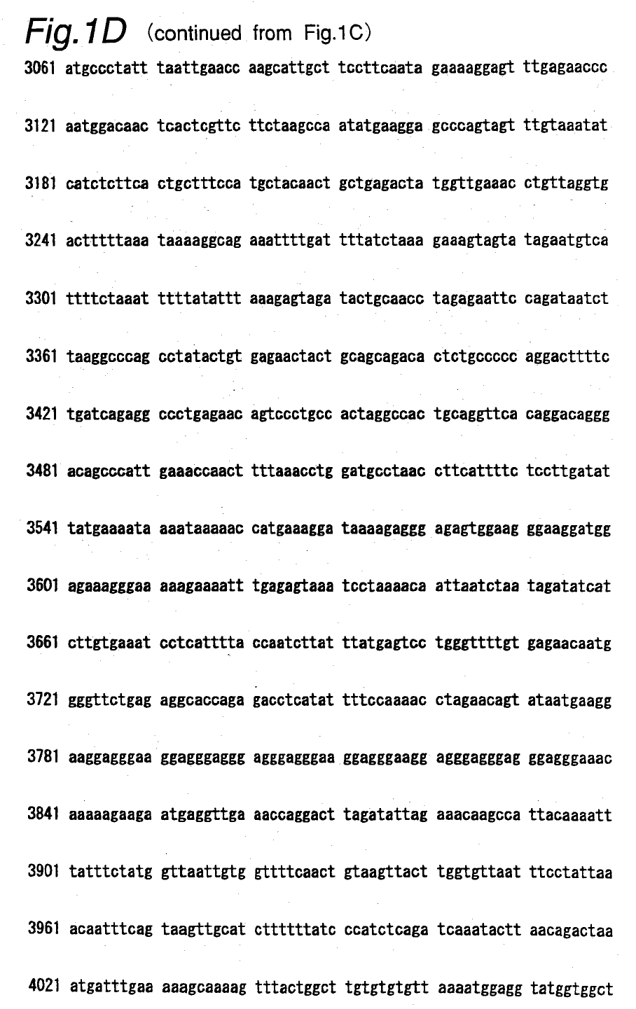

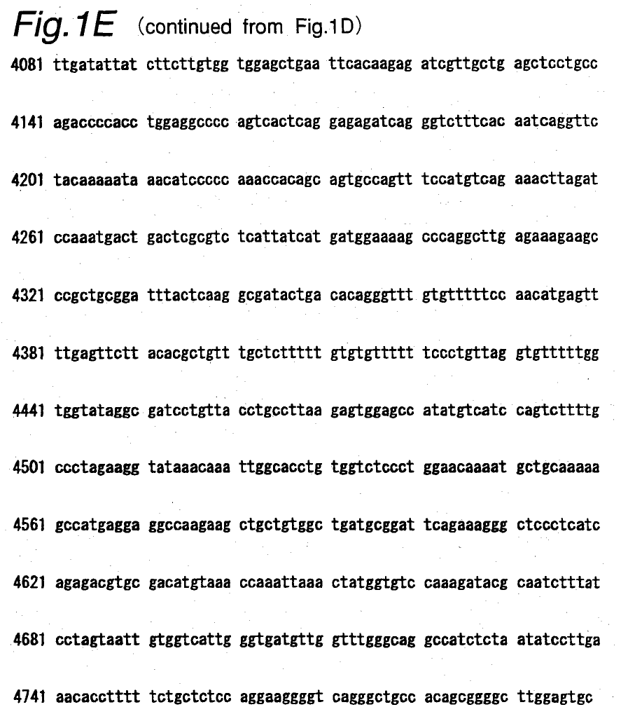

That is to say, total DNAs were extracted, and the nucleotide sequence of the hBD-2 promoter region was directly sequenced with an ABI PRISM 310 Genetic Analyzer (Applied Biosystem, CA, USA) (FIGS. 1A to 1E) to identify mutations (Table 1) Then, a luciferase enzyme activity assay (through the use of a luciferase expression vector) was carried out to examine whether mutation in this region is involved in the depletion of the transcription of hBD-2. The promoter sites in KB cell were recovered from mutation by GeneEditor in vitro Site-Direct Mutagenesis. The changes of the luciferase activity were compared between non-recovered and recovered KB cell promoters. The luciferase activity corresponding to the promoter activity was increased by recovery from mutation. Further, the binding abilities of a nuclear protein (trans factor) to each mutation site were analyzed by a gel shift assay, using a nuclear extract from cells. According to direct sequencing, it was revealed that mutants are present in sites of 4 regions. Such regions include cis-elements such as C/EBPβ, TATA box and Sox5. Moreover, the oligonucleotides probe having a mutation site exhibited a decreased binding affinity with the nuclear extract from KB cells. These results indicate that the mutation on the sequence of the hDB-2 gene promoter is involved in the depletion of the expression of hDB-2 mRNA in KB cells.

In the non-inflammatory, normal gingiva, β-defensin, especially human β-defensin 2 (hBD-2) is expressed constitutively and is regarded to constitute a part of the normal barrier action of the oral epithelium. However, if a gene mutation occurs in the hBD-2 promoter region, there is a possibility that the expression of hBD-2 does not sufficiently elevate against the invasion of pathogenic bacteria.

(B) Method of Estimating Susceptibility to Periodontal Disease

The above described findings demonstrate that the insufficient expression of the defensin gene is closely associated with the onset of periodontal diseases or thereby affected conditions. Accordingly, if the possibility that the insufficient expression may occur could be successfully estimated by detecting changes at the causative gene level, specifically, changes in promoter functions evoked by mutant base induced by gene mutation, it becomes possible to predict the potential onset and development of periodontal disease. Such an influence of a gene mutation can also be analyzed by measuring the transcriptional activity of a gene in a cell or tissue, e.g. the expression level of mRNA transcribed in a cell, by Northern blotting using a DNA probe However, mRNA level has only a subsidiary meaning as data for estimating susceptibility to periodontal disease.

The method of the present invention is a method of collecting data for estimating susceptibility to periodontal diseases, which focuses attention on the regulation of the expression of the defensin gene. Specifically, the method of the present invention is characterized in that it comprises: in order to detect the presence of a gene mutation and/or a mutation site existing in the promoter region of a human defensin gene in a sample, by using a nucleotide sequence being a part of the promoter of the defensin gene and comprising a mutant base as a nucleotide sequence for a probe, determining

(i) the degree of hybridizing ability in hybridization between the defensin gene promoter nucleotide sequence in the sample and the above probe, and/or

(ii) an amplification ability in gene amplification where primers comprising the nucleotide sequence of the above probe are used;

thereby clarifying the change of the activity of the defensin promoter to regulate the expression of the defensin gene based on the thus detected presence of gene mutation and/or a mutation site.

The method of the present invention, which combines homology with a detection probe, the detection of a gene mutation in the promoter region of the defensin gene and susceptibility to periodontal disease together, is based on the following principle. If DNA in a sample collected from a subject, or amplified defensin gene-derived DNA or a fragment thereof, is fully hybridized with a detection probe comprising a mutant sequence, or if gene amplification is well carried out using primers having the nucleotide sequence of a detection probe, it means that the above DNA or fragment thereof has high homology with the detection probe in nucleotide sequence. In such a case, there is a high possibility that the sample DNA may comprise a sequence similar to the nucleotide sequence of the detection probe, and therefore a gene mutation may occur in the defensin gene promoter region. If a mutant base is comprised in the promoter region which functions to down-regulate the expression activity of defensin, the expression is not normally effected out, and such insufficient expression exerts an influence on the generation and secretion of defensin, thereby bringing an increased risk of susceptibility to periodontal disease because defensin cannot intervene in the activity of the disease-related bacteria.

Any defensin can be used as long as it belongs to the human defensin family. Specifically, defensin maybe either α-defensin or human β-defensin, but β-defensin is preferable.

The detection of the mutation of a nucleotide sequence exerting a possible influence on the promoter activity can be carried out extremely simply and promptly by employing the above described method, because this method does not require to determine the entire or a part of the sequence of a corresponding gene. This is one of features of the present invention, which should be emphasized.

(C) Human β-Defensin 2

The above described method will further specifically explained below, taking human β-defensin 2 as an example.

Sample

As an object to which the present invention is applied, any sample can be used as long as it comprises nucleic acid collected from tissues such as mucous membrane, gingival tissues in the oral cavity or the like. Specific examples of such a nucleic acid-containing sample include a cell homogenization liquid, tissues such as an epithelial cell and a mucous membrane, blood and others.

The above blood may be ordinary peripheral blood, arterial blood, venous blood, or a buffy coat (a leukocyte fraction) that is a formed ingredient, obtained by performing centrifugal separation or the like on these types of blood. Where whole blood or a formed ingredient of blood is used as a sample, it is preferable to perform a hemolysis procedure.

Separation of Genes or DNAs

DNAs can be separated from a sample and purified by phenol-chloroform extraction and ethanol precipitation according to the conventional methods. It is generally known that concentrated chaotropic reagents such as guanidine hydrochloride and isothiocyate which have an almost saturation concentration are used to release nucleic acid. These chaotropic reagents effect the release of nucleic acids by denaturing and/or solubilizing protein. Since these chaotropic reagents are used at a high concentration almost equivalent to their saturation concentration, many soluble ingredients such as protein, other than released nucleic acid are contained in the obtained nucleic acid-released solution.

Accordingly, unless the obtained nucleic acid-released solution is purified again to remove soluble protein or the like, a nucleic acid amplification method which is based on enzyme reactions such as PCR (Polymerization chain reaction) cannot be used. Examples of a purification method applied to the nucleic acid-released solution include a method of precipitating nucleic acid by adding ethanol or isopropyl alcohol, a method of allowing nucleic acid to be adsorbed to silica beads, ultrafiltration, column chromatography and others.

There is a simple and quick method, which does not apply the above phenol-chloroform extraction, but instead, directly treats a sample with a protease solution containing a surfactant (Takashi Saito, “PCR Experiment Manual” HBJ, 1991, p. 309).

Amplification and Fragmentation of Genes

A defensin gene of interest, a promoter thereof or DNA fragments thereof can efficiently be obtained from the extracted genes, DNAs or a mixture of fragments thereof by performing amplification by PCR and screening with a DNA probe described later. As primers used in amplification, all of 4 primer sets or at least one primer set described later may be used as appropriate. Usually, in each of these primer sets, two oligonucleotides whose nucleotide sequences are shown are used together.

Where the obtained genomic DNAs or genes are too large, the DNAs or genes may be fragmentized by means of the conventional method using suitable restriction enzymes such as BamHI, BgLII, DraI, EcoRI, EcoRV, HindIII and PvuII. DNAs used as a sample for collecting data for estimating susceptibility to periodontal disease and an aggregate of DNA fragments can be prepared by the combination of screening and amplification.

Each of FIGS. 1A to 1E shows the nucleotide sequence of the human β-defensin 2 gene. The nucleotide sequence comprises a promoter region in addition to a coding region (staring from position 2732). It is possible for a person skilled in the art to prepare and set primers to be used in amplification, probes to be used in screening and others by chemical synthesis or the like on the basis of such a given nucleotide sequence.

Detection of Gene Mutation in Defensin Gene Promoter

Detection of the presence of some abnormality in a regulatory gene involved in expression regulation is an effective approach to analyze the change of the regulation of defensin expression. Specifically, it is desirable to analyze whether a mutation exists in a region upstream of the coding region of the human defensin gene, that is, the mutation exists on the nucleotide sequence in the promoter region. Each of FIGS. 1A to 1E shows the nucleotide sequence of the human β-defensin 2 gene. The nucleotide sequence comprises both of a promoter region and a coding region (starting from position 2732).

The presence or absence of a mutation of the human β-defensin 2 gene was examined using 9 types of the established cell lines (cell lines), namely, KB cells; a human nasopharyngeal carcinoma-derived established cell line, SCC-9; a human lingual cancer-derived established cell line, SAS; a human lingual cancer primarily originated foci-derived established cell line, HSC-2; a human mouth-floor tumor-lymph node metastatic foci-derived established cell line, HSC-3; a human lingual cancer-lymph node metastatic foci-derived established cell line, HSC-4; a human lingual cancer-lymph node metastatic foci-derived established cell line, Ca9-22; a human mandibular gingival cancer primarily originated foci-derived established cell line, OSC-19; a human lingual cancer-cervical metastatic foci-derived established cell line OSC-20; and a human lingual cancer-cervical lymph node metastatic foci-derived established cell line).

Initially, using the above 4 types of primer sets 1 to 4, a nucleotide sequence in a region upstream of the coding region of the defensin gene was amplified. Subsequently, a gene mutation was examined in each of the amplified areas. To detect the gene mutation, two types of methods were employed such as direct sequencing and SSCP (single strand conformational polymorphism) which examines the presence or absence of a gene mutation, utilizing the difference of mobility brought about by a slight difference in the sequence of a single strand DNA in electrophoresis.

As a result, although frequency is varying depending on samples, in the area amplified by primer set 1, G was substituted by C at position −1431, and in the area amplified by primer set 2, G was substituted by T at position −1035, A was substituted by G at position −1027, G was substituted by A at position −936, C was substituted by T at position −923, T was substituted by C at position −912, and G was substituted by A at position −874. In the area amplified by primer set 3, C was substituted by T at position −539, and A was substituted by G at position −472, and in the area amplified by primer set 4, T was substituted by C at position −108.

Of these, mutations occurring at position −1035 to at position-472 most likely inhibit the expression of β-defensin 2. These mutations have a significant impact on the gingiva because these mutations reduce the antibacterial action of the gingiva, enhancing the risk of susceptibility to periodontal disease.

Thus, it was revealed that gene mutations exist in the defensin gene promoter region.

In addition, some of the regions containing these mutations encompass the so-called cis-regions and thus such mutations are presumed to affect significantly the control of transcription.

Accordingly, the detection of such mutation sites enables to find the strength of the antibacterial action of defensin or the like in human oral cavity and to estimate susceptibility to, or a risk of contracting a disease caused by bacterial pathogen such as periodontal disease.

These gene mutations, as described below, can be detected by analyzing the level of matching in hybridization or an ability of the amplification of a gene in gene amplification.

Preferred Embodiments for Carrying Out the Invention

(A) Detection Probe

A probe used in the above described detection of a gene mutation is within the scope of the present invention and forms another aspect.

To efficiently detect a DNA of interest, a molecule comprising a partial nucleotide sequence of the target DNA and hybridizable therewith can preferably be used as a “detection probe.” This is based on the complementarity between the nucleotide sequence of the probe and the sample DNA strand. Generally, the suitable probe can be designed by utilizing the conventional techniques from a nucleotide sequence, which comprises one or more nucleotide sequences complementary to the nucleotide sequence of a promoter that is a regulator of defensin, or is substantially homologous thereto, according to known techniques in the art.

Such a nucleic acid molecule can be used as a PCR primer or a hybridization probe for detecting a portion having substantial homology.

The “hybridization prove” herein denotes a DNA fragment containing a partial sequence in the promoter region of the defensin gene, being used for detection by hybridization.

The “substantially homologous” sequence includes a sequence having approximately 50% or more, e.g. 60% or more sequence homology to relevant modified sequences comprising a substitution, addition and/or deletion of one or more bases.

The promoter which is the object of the present invention, includes 10 types of mutant promoters comprising a substitution of bases as well as a normal promoter. The direct use of the nucleotide sequences of these promoters is not practical because these sequences per se are too long and large to be used as a probe. Therefore, an oligonucleotide which is homologous to a part of the sequence of the promoter is used instead. The oligonucleotide used as a probe may comprise at least 5 nucleotide sequence portions being each of upstream and downstream from a mutation site in the promoter. To ensure specific hybridization, the oligonucleotide desirably comprises sequence portions of 10 or more nucleotides chain length, preferably 15 or more nucleotides chain length, respectively.

This oligonucleotide used as a probe can easily be prepared by chemical synthesis using a DNA synthesizer Moreover, the addition of a marker for detection after hybridization to the oligonucleotide comprising a part of the promoter sequence, enables to increase detection sensitivity when the oligonucleotide is used. Thus, such oligonucleotide with a marker can preferably be used as a probe for hybridization. Otherwise, the oligonucleotide is appropriately modified so that it can be used as a primer for gene amplification. A nucleic acid molecule hybridizable with a sequence substantially homologous to or functionally equivalent to the sequence of the above probe, is also included in the scope of the present invention.

An oligonucleotide used to detect a mutant type sequence comprising a mutant base in the promoter region of the human β-defensin 2 gene, has the following sequence. Of the promoter nucleotide sequences, preferred examples of a nucleotide sequence, wherein preferably it comprises sequences of at least 5 nucleotide being each of upstream and downstream from a mutation site or at-least-10-nucleotide-containing sequences of which 3′ terminus is the nucleotide of a mutation site, include the following nucleotide sequences of the present invention:

a DNA nucleotide sequence (a sequence portion of bases 1176 to 1606 in the nucleotide sequence in FIG. 1B) amplified by primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

wherein G is substituted by C at a site −1431 (mutation site 1) located upstream of the transcription initiation point of the human β-defensin 2 gene, and/or

a DNA nucleotide sequence (a sequence portion of bases 1561 to 2031 in the nucleotide sequence in FIG. 1B) amplified by primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

wherein G is substituted by T at a site −1035 (mutation site 2-1), A is substituted by G at a site −1027 (mutation site 2-2), and/or G is substituted by A at a site −936 (mutation site 2-3), and/or C is substituted by T at a site-923 (mutation site 2-4), and/or

a DNA nucleotide sequence (a sequence portion of bases 1561 to 2031 in the nucleotide sequence in FIG. 1B) amplified by the same primer set as above,

wherein T is substituted by C at a site −912 (mutation site 2-5), and/or G is substituted by A at a site −874 (mutation site 2-6), and/or

a DNA nucleotide sequence (a sequence portion of bases 1987 to 2470 in the nucleotide sequences in FIGS. 1B and 1C) amplified by primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

wherein C is substituted by T at a site −539 (mutation site 3-1), and/or A is substituted by G at a site −472 (mutation site 3-2), and/or

a DNA nucleotide sequence (a sequence portion of bases 2421 to 2760 in the nucleotide sequence in FIG. 1C) amplified by primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

wherein T is substituted by C at a site −108 (mutation site 4).

These nucleotides used as probes can be chemically synthesized using a DNA synthesizer. To use these nucleotides as hybridization probes or primers for PCR amplification, markers for detection and/or amplification are preferably attached thereto according to conventional methods, and these nucleotide sequences with markers may be further modified into a form which is easy to handle.

Generally, a DNA probe can be labeled by 5′ End Labeling, the Nick Translation method, the random primer method and others, using a radioactive isotope detection marker, or more preferably, a non-radioisotope detection marker such as a fluorescent dye or a chemoluminescence dye. More specifically, detection with high sensitivity can be realized by a chemoluminescence method of labeling with alkaline phosphatase or horseradish peroxidase (HRP), or by a chemical fluorescence method of using fluorescence labeling with Phycoerythrin or fluorescein (FITC).

Preferably, a technique of immobilizing the above probe on a solid phase supports is generally employed to efficiently separate and detect the DNA hybridized with the probe. For example, a probe can be immobilized by biotinating the probe with a suitable detection marker and then binding it to streptavidin which is attached to a suitable solid phase supports such as beads, a plate, a sheet, a membrane or a filter. The use of magnet beads as beads makes the operation more convenient.

In the present invention, in addition to the above probe, a “DNA probe” may also be used as necessary. Such DNA probe is also encompassed in the concept of the above detection probe. The DNA probe is used to search a DNA of interest from a genomic library or DNA library by hybridization. Specifically, the DNA probe is a DNA fragment having a nucleotide sequence complementary to the sequence of the promoter of defensin or a derivative comprising the same, so that the DNA probe can hybridize with the promoter of defensin. However, the DNA probe and a hybridization probe may comprise the same nucleotide sequence, or those nucleotide sequences may partially be overlapped.

This DNA probe is used when a targeted DNA is specifically searched from a genome library or a DNA library by use of hybridazation. According to the present invention, the DNA probe is used when a DNA comprising the defensin gene is specifically searched from a DNA mixture obtained from a sample.

It is obvious for a person skilled in the art to prepare the above DNA probe (a detection probe, a DNA probe, etc.) on the basis of the nucleotide sequences shown in FIGS. 1A to 1E according to known techniques such as chemical synthesis with a DNA synthesizer or a gene amplification technique by PCR and to set a nucleotide sequence useful as a probe.

(B) Collection of Data for Estimation of Susceptibility to Periodontal Disease

Preparation of DNA Sample

Genes or DNAs are extracted from tissues containing cells as described above, and then they are amplified and screened. The obtained DNA and the DNA fragment mixture thereof are used as samples for the following analysis. If the amount of the gene or DNA is too small and insufficient, or if the amount of the target DNA (which is derived from the defensin gene promoter region and comprises the sequence of the mutation site) is small and insufficient for analysis, the gene or DNA maybe amplified as appropriate. In contrast, if the obtained DNA is too large, it may further be fragmentized with suitable restriction enzymes.

Preparation of Evaluation Data for Prediction by Hybridization

To analyze the homology between the above hybridization probe comprising a gene mutation in the human defensin gene promoter region and the DNA in the human defensin gene promoter region contained in the target DNA sample (the DNA and the fragment mixture thereof prepared from a specimen obtained from a subject whose susceptibility to periodontal disease is to be examined), the following Southern hybridization is employed.

Southern Analysis Hybridization

A labeled “hybridization probe” is hybridized with the target DNA (DNA derived from the defensin gene promoter region and comprising the sequence of the mutation site) in a sample placed on a suitable analysis plate, slide or nitrocellulose membrane under stringent conditions. Otherwise, the sample DNA may be reacted with the target probe which has previously been immobilized to a plate, chip or the like. Hybridization can be carried out on a suitable solid phase carrier such as a cellulose membrane, nitrocellulose membrane or nylon membrane filter.

The term “stringent conditions” is herein used to mean conditions where a specific hybrid is formed and non-specific hybrid is not formed (in other words, it is conditions where cross-hybridization with a polynucleotide having a low sequence homology does not significantly occur). The specific hybridization denotes that a great majority of “hybridization probes” correctly form base pairs of a typical Watson-Crick model type with the complementary sequence of the target DNA. In this case, if a portion exists where the mismatching of base pairs is brought about by the mutation of a base, hybridization should hardly occur. It is generally difficult to clearly show these conditions with certain figures, and therefore individual conditions (e.g. a reaction temperature, a salt concentration, etc.) are specifically set in an actual system.

Examples of such conditions include: conditions where DNAs having high sequence homology, for example, having 90% or more homology, are hybridized with each other but DNAs having sequence homology lower than 90% are not hybridized; and conditions where hybridization is carried out in a SSC salt concentration corresponding to the ordinary “washing” condition in Southern hybridization (SSC is an abbreviated form of Standard Saline Citrate and means conditions of 0.15 M NaCl, 0.015 M sodium citrate, pH 7.2).

Conditions which should especially be considered are temperature, anion concentration and others. As a reaction temperature, a temperature, 15° C. to 25° C. lower than the DNA melting temperature, is generally considered to be the optimal temperature. Nevertheless, since the formation of a hybrid is influenced by the type or length of nucleic acid, preferred conditions are required to be set for individual properties. Moreover, the formation of a hybrid is influenced also by anion concentrations, for example, the concentration is set within a range of 0.15 M to 1 M in sodium chloride concentration.

Southern hybridization is carried out by performing agarose gel electrophoresis on the DNA fragments obtained by cleaving a DNA with restriction enzymes and/or the DNA collected from the sample (polyacrylamide gel electrophoresis is more suitable for a fragment of 1 kb or shorter), and then transferring the DNA on a gel to a membrane filter (mainly, a nitrocellulose filter). This time, the gel is treated with alkali (e.g. sodium hydroxide) so that the DNA is also denatured in the gel. The DNA is transferred from the gel to the filter and fixed thereto by aspiration with an aspirator or the like. The above described hybridization probes having suitable labels are applied on the DNA. Then, hybridization probes which are still unbound are removed by washing the filter.

The amount of the hybridized probes is quantified by analysis with a label (marker). Where the probes are labeled with a fluorescent dye, the fluorescence intensity of the probes may be measured by using a scanner with a laser beam or the like and converted into numeric character data. The probes labeled with a chemoluminescence dye are quantified in the same manner as described above, that is, the light intensity of the probes may be measured and converted into numeric character data. Where the probes are labeled with a radioactive isotope, the binding amount is determined by autoradiography or using a scintillation counter. As a control, a non-mutated DNA may be used.

Degree or strength in the formation of a hybrid between the target DNA (i.e. the DNA containing a sequence of mutation derived from the promoter region of the defensin gene) in the sample and the above hybridization probe comprising a gene mutation in the human defensin gene promoter region, is indicated by the scale or strength of the hybridization, which is obtained by detecting specific signals by detection means, converting the signals into the electrical intensities and finally converting the intensities into numeric values. On the basis of the thus obtained scale or strength of the hybridization, the target DNA in the sample is broadly divided into the below-mentioned three groups. Since this hybridization analysis is based on the indication of homology between the target DNA in the sample and hybridization probes, differing from a cumbersome method of directly determining nucleotide sequences and comparing them, this analysis enables simple and quick detection of the presence of a mutation in the promoter region. Since the presence or absence of a mutation in the promoter region affects the expression activity of defensin, future susceptibility to periodontal disease may be predicted on the basis of such detection.

(i) Low-Risk Type

The target DNA (derived from the defensin gene promoter region and comprises the sequence of the mutation site) is hardly hybridized with the above described hybridization probe comprising a gene mutation in the human defensin gene promoter region, but it is hybridized with a hybridization probes having a normal type-nucleic acid sequence. Such a normal nucleotide sequence is considered to have almost no mutation in the promoter region. In this case, defensin is normally expressed in the future, and the target DNA is therefore categorized as a low-risk type.

(ii) High-Risk Type

The target DNA is hybridized with any one of the above described hybridization probes comprising a gene mutation in the human defensin gene promoter region. This target DNA is considered to have some mutations in the promoter region, and there is a high possibility that defensin is not normally expressed in the future. Therefore, this target DNA is categorized as a high-risk type.

(iii) Undetermined Type

In some cases, the target DNA is not substantially hybridized not only with the above described hybridization probe comprising a gene mutation in the human defensin gene promoter region, but also with a hybridization probe having the normal type-nucleotide sequence. That is, with no information provided regarding mutations in the promoter region, this type of the target DNA cannot be categorized into either the above (i) or (ii).

Collection of Evaluation Data for Prediction by Amplification Method

A primer having a sequence complementary to a template DNA is used in the gene amplification method. Accordingly, the efficiency of gene amplification greatly varies according to the types of primers. Focusing on this property, a primer comprising a nucleotide sequence comprising a mutant base in the defensin gene promoter is prepared. The above-mentioned DNA probes may be employed for the preparation of such primer. Gene amplification is carried out by the gene amplification method that utilizes the primers prepared and a DNA sample obtained from a specimen (the DNA prepared from a specimen obtained from a subject whose susceptibility to periodontal disease is to be examined, and the DNA fragment mixture thereof) as a template DNA. Evaluation data for the prediction can be obtained from the amplification results. Specifically, amplification is carried out using primers comprising any sequence of mutant types which were found. When a DNA sample having a normal sequence is used, the complementarity between a template DNA of a normal type and a mutant primer is less than that between a template DNA of a normal type and a primer having a normal sequence, and the efficiency of amplification is thereby decreased. There is a common PCR technique for analyzing gene mutation (S. Kwokm et al., Nucl. Acids Res. 18:999-1005, 1990).

For detection of a gene mutation, besides utilization of the above-mentioned primers incorporating a mutation sequence, one may alternatively perform an allele specific PCR in which the efficiency of amplification is examined by using another type of primers.

A chromosome gene is originated from parent's gene, consisting of a maternal gene and a paternal gene. The term “home” refers to a gene consisting of the same type of genes derived from parents while “hetero” refers to a gene consisting of a different type of genes from parents. Among genotypes, the genotype appearing with higher frequency is called hereinafter “allele 1” (normal type), and the genotype appearing with lower frequency is called “allele 2” (mutant type). A genotype-specific PCR reaction, i.e. an allele specific PCR reaction can be performed wherein genes of “allele 2” (mutant type) participate in the reaction, but genes of “allele 1” (normal type) do not, or otherwise entirely vice versa. Which type the gene in question belongs to either of allele 1 or allele 2 can be deduced from the observation whether or not a high efficiency of amplification is attained in such a PCR.

In the above-mentioned allele specific PCR, 3 types of primers are used in combination. Specifically, the primers are a common type primer (common primer) specific to both of “allele 1” (normal type) and “allele 2” (mutant type), and two types of primers, i.e. allele-specific primers specific to either of the alleles.

The allele-specific primer is required to comprise an oligonucleotide containing mutation sites of “allele 1” (normal type) and/or “allele 2” (mutant type), more preferably an oligonucleotide which terminates with a mutation site of “allele 1” (normal type) and/or “allele 2” (mutant type) at its 3′terminus. Each of the oligonucleotides may have preferably around 10 to 30 bp in length. For discriminating “allele 1” (normal type) from “allele 2” (mutant type) with a greater accuracy, it is favorable that the primer for detecting “allele 2” (mutant type) should be longer than that for detecting “allele 1” (normal type).

The examples of the primers include:

Primer Set for Detection of −1027 Position

common primer

5′ GATGGGAAACACTTATGAAGG 3′

primer detecting a mutation-type

5′ GGCCGCCAATTGCTACTCTGGGTTACGGAG 3′

primer detecting a normal-type

5′ TTGCTACTCTGGGTTACGGAA 3′

Primer Set for Detection of −912 Position

common primer

5′CTTTGGGAGCCAGGGCTTTCT 3′

primer detecting a mutation-type

5′ AGGGAGACCACGTGGAGGCCTTGCAGCCCC 3′

primer detecting a normal-type

5′ ACGTGGAGGCCTTGCAGCCCT 3′

Primer Set for Detection of −874 Position

common primer

5′CGTCACTCAGGGAATGTCAGC 3′

primer detecting a mutation-type

5′CTTCTCCACCAAATCCCAAGGGCAGTGACA 3′

primer detecting a normal-type

5′CAAATCCCAAGGGCAGTGACG 3′

In the allele specific PCR method, PCR may be typically performed by using together one common primer and two types of primers having, at their 3′ terminus, a nucleic acid of either mutant-type base or normal-type base. In the case of “hetero”, the same amplification efficiency will be obtained by using any of the two types of the primers, while in the case of “homo”, the efficiency will be decreased if using either of the two primers.

Measuring the degree of amplification using such primers by employment of “Lightcycler” (Roche Diagnostic Inc.) may enable one to discern homo- and hetero-type of the DNA sequence of a gene.

Thus, according to the allele specific PCR method, homo- and hetero-type of genes can be very quickly discriminated from each other to thereby enable an advanced gene analysis in a shorter time for assessment of an antibacterial activity with more accuracy, which will constitute a preferred embodiment from another aspect of the present invention.

Examples of a method of performing a nucleic acid amplification test (NAT) include the PCR (Polymerization chain reaction) method of Roche, the TMA (Transcription Mediated amplification-hybridization protection assay) method of Gen-Probe, Inc., the LCR (Ligase chain reaction) method of Abott, the LAMP (Loop-mediated isothermal amplification of DNA) method of Eiken Chemical Co., Ltd., the ICAN (Isothermal and chimeric primer-initiated amplification of nucleic acid) method of Takara Shuzo, and others.

Generally, an oligonucleotide comprising around 15 to 40, preferably around 15 to 30 nucleic acid bases, is used as a primer. The oligonucleotide used as a primer can easily be prepared by a chemical synthesis method that utilizes a DNA synthesizer. Examples of the preferred primers of the present invention include sequences amplified by any one of the above described primer sets 1 to 4 comprising mutation sites, and these primers may appropriately be used as primers for the analysis of gene mutation based on PCR techniques. (It should be noted that generally, in these primer sets, two oligonucleotides whose sequences are shown in a juxtapositional manner above are used together.) The primer sets and oligonucleotides having these nucleotide sequences are used as primers and included in the scope of the present invention.

Specific operations in the gene amplification can routinely be carried out by a person skilled in the art according to instructions provided from manufacturers or, if necessary, by setting preferred conditions. The level of DNA amplification is determined by subjecting the amplified product to agarose gel electrophoresis and detecting DNA with fluorescence such as ethidium bromide. The presence or absence of amplification may simply be confirmed by the visual inspection of the concentration of electrophoretic bands. More precisely, the gel is scanned with a densitometer to convert the level of amplification into a numerical value. For such analysis, “Lightcycler” (Roche Diagnostic Inc.) should be mentioned to be a useful PCR apparatus, in that it is capable of demonstrating the presence or absence of amplification or evaluating the degree of the amplification numerically when the amplification reaction is performed.

As is the case with the above described hybridization, when DNA is highly amplified by using an oligonucleotide comprising a mutant nucleotide as a primer, the target DNA (i.e. the DNA containing a sequence of mutation derived from the promoter region of the defensin gene) is well compatible with the primer. In other words, such high complementarity indicates high sequence homology between the target DNA and the oligonucleotide of the primer. Comparison of amplification efficiency enables to detect mutant promoter DNAs in the sample. Since the presence of such mutants is associated with the expression activity of defensin, the risk of future susceptibility to periodontal disease can be predicted based on the presence of mutants. The level of the presence of mutants is classified into the following two types for convenience.

(i) Low-Risk Type

In this case, when a primer comprising a nucleotide sequence containing a gene mutation in the human defensin gene promoter region is used with the template DNA derived from the sample DNA, gene amplification is not substantially carried out, or the level of gene amplification is low. This is because the nucleotide sequence in the sample, that is, the template DNA, is not well compatible with the primer comprising a mutant sequence. Accordingly, it is considered that almost no mutants exist in the promoter region of the sample gene and that defensin is normally expressed in the future. Therefore, this case can be categorized as a low-risk type.

(ii) High-Risk Type

In contrast to (i) above, when a primer comprising a nucleotide sequence containing a gene mutation in the human defensin gene promoter region is used with the template DNA from the sample, gene amplification is fully carried out. This is because the nucleotide sequence in the sample, that is, the sample DNA, is well compatible with the primer comprising a mutant sequence. Conceivably, some mutation exists in the promoter region of the sample gene and defensin is unlikely to be normally expressed in the future. Therefore, this case can be categorized as a high-risk type.

Mixing Method

The methods of collecting data for estimating susceptibility to periodontal disease are not necessarily limited to the above two methods. A method of detecting and identifying a gene mutation on a DNA sequence of interest by the combined use of the hybridization method and the PCR gene amplification method can also be employed. The details of this method are disclosed in Japanese Patent Laid-Open No. 2001-57892.

Moreover, a method which comprises screening a DNA having a mutation site in the sequence by the use of DNA probes, then amplifying the DNA by PCR method using primer set 1 to 4, and subjecting the obtained amplified product to gel electrophoresis so as to examine the presence or absence of a mutation by the difference of the mobility of the DNA, can also be used.

Estimation

Susceptibility to periodontal disease is estimated on the basis of the data obtained by the above-described methods. The data used herein may be those obtained by any one of the above methods, or data obtained by the combined use of the above methods. The estimation of susceptibility to the disease can efficiently be carried out using the following data processing system.

Estimation of a risk of susceptibility to periodontal disease is eventually determined by dentists' comprehensive review of the above obtained data, other clinical data, and the conditions of individual subjects (e.g. age, sex, anamnesis, the presence of dental caries, the condition of the gingiva, life habit, etc.) The prediction made based on the above comprehensive review has an increased confidence.

(C) Test Reagents

The kit for estimating susceptibility to periodontal disease of the present invention is characterized in that it comprises, as a probe, at least one type of nucleotide sequence selected from at least the above-described nucleotide sequences for probes, and further comprises the above primers as necessary, so as to detect a mutant type sequence in the human defensin gene promoter region.

Specifically, the present invention provides a kit including various materials necessary to carry out the above detection method, that is, probes for carrying out hybridization, reagents and/or primers necessary to carry out gene amplification, and other reagents.

Such a kit may further include other reagents, test material and so on in combination, as necessary. For example, the kit may further include, as kit elements, reagents used to separate nucleic acid from a sample, a microtiter plate, materials for hybridization, materials for PCR and others. Moreover, these reagents may include, primers, various enzymes, buffers, washing pollutions, lytic solutions and others in addition to the above probes. Furthermore, the kit may further include an electrophoresis device and detection means. However, in general, general-purpose devices, which have already been used, are conveniently used.

DNA Chip

Both the above described hybridization and the gene amplification method are methods involving a multi-step analysis accompanied with many processes. Accordingly, in order to uniformize conditions of processing, analyzing and detecting specimens, to solve problems on the control of test precision such as errors of measurements or variations of operations and problems on degeneration or contamination, and further, to quickly and simply carry out processing, analysis and detection, the collection of data for estimation of susceptibility to the disease is desirably carried out using a DNA chip in which all these operations are conducted on a single plate. Thereby, the variations of data or like are prevented and the precision of data is also improved. The use of the above described kit enables to solve these problems to a certain extent, but if the DNA chip is used, problems of the kit involving complicated procedures of using a large number of reagents and devices are overcome.

The present invention provides a DNA chip wherein it comprises, as a probe, at least one type of nucleotide sequence selected from the above-described nucleotide sequences for probes, and further comprises the above primers as necessary.

A particularly preferred embodiment of the DNA chip includes a DNA chip characterized in that operations from the separation of nucleic acid to the generation of detection signals can be carried out in a single carrier by incorporating the essential reagents and ingredients from the above kit in the carrier and applying a specimen in a hole in a certain compartment allocated on the chip apparatus. That is to say, in the method of the present invention, it is not necessary to fix a large number of DNA fragments on a solid phase substrate for a profile of gene expression, but it is rather desired that the processes of extracting and purifying DNA from a sample, and a DNA amplification process are all incorporated into a chip. Preferably, these processes, hybridization and/or amplification reaction processes and a detection process are wholly incorporated into a single chip. As such a DNA chip, either an Affimetrix-type DNA chip or a Stanford-type DNA chip can be used.

In another embodiment, the DNA micro-array or DNA chip can also be used to screen for multiple diseases. Since originally a DNA chip is a tool for handling a large number of DNAs (several thousands to several ten thousands) and processing a large-capacity data, the application of the DNA chip for genes associated with not only β-defensin 2 but also a plurality of diseases enables an inclusive monitoring of the expression of the gene associated with multiple diseases through the analysis of whole information on expression of many genes.

Data Processing System

The system of the present invention captures, as data, numerical values converted from signals obtained from the above kit or DNA chip, prepares a file, and stores it in a certain directory on a computer. This system can statistically process the numerical data, analyze the activity of the human β-defensin 2 promoter which regulates the expression of β-defensin 2, and estimate susceptibility to periodontal disease. The data processing is carried out using a preferred software capable of making necessary amendments, normalization and statistical analysis. A person skilled in the art may establish a system for dealing with such a data processing by use of conventional techniques, protocols and procedures.

The data may be continuous data or discrete qualitative data. The data is processed by an appropriate statistical method. A method which can be carried out most simply and easily is a comparison with the case of a normal nucleotide sequence, based on the use of the normal nucleotide sequence as a control.

The precision of the onset probability can be improved by increasing the confidence of correlative index by the integration of relevant clinical data. If the ratio proving true as to the disease onset is at lowest 50%, favorably around 70%, this is generally considered to be sufficiently practical at a clinical scene.

According to the present invention, gene mutations have been identified in a defensin gene promoter regulating the expression of defensin, a key substance for periodontal disease. Based on the identification, a method is developed enabling to simply and quickly determine the activity of the promoter at low cost.

Since the activity to express defensin is estimated based on the detection of a mutation in the defensin gene promoter region, the future susceptibility to periodontal disease can be predicted with high precision.

The use of the method of the present invention enables to realize a tailor-made thereby from the view point of the prevention of periodontal disease.

### EXAMPLES

The present invention will further be explained in the following examples. However, the examples are not intended to limit the scope of the invention.

### Example 1

Detection of Gene Mutation in β-Defensin 2 Promoter Region by Direct Sequencing

DNA was extracted from the above described 9 types of cultured cell lines. The extraction of DNA from the cultured cells was carried out using Sepa Gene (Sanko Jyunyaku). Using AmpliTaq Gold Master Mix (ABI PRISM), the extracted DNA was amplified with Thermalcycler (TaKaRa). Oligonucleotides having sequences comprising mutation sites, which are around 40 nucleotide sequences as their cores (that is, the above described 4 types of primer sets), were used as primers. Thereafter, electrophoresis was carried out using SeePlaque agarose (1.5% Agarose gel) (BMA), in which bands were cut off later. The gel was dissolved at 65° C., and amplification by PCR was carried out again by the same method. The obtained PCR product was again subjected to electrophoresis with NuSieve 3:1 agarose (1.5% Agarose gel) (BMA) and the bands were cut off, followed by the extraction of DNA with QIAEX II Gel Extraction Kit (QIAGEN) Then, the extracted DNA was reacted using Big Dye Terminators Cycle Sequencing Ready Reaction Kit (ABI PRISM). Subsequently, the DNA was purified using a column, subjected to vacuum-freeze drying, and then dissolved in formamide (ABI PRISM). Heat denaturation was carried out at 95° C. for 5 minutes followed by quenching, and thereafter sequencing was carried out with an autosequencer, ABI 310 Genetic Analyzer (ABI PRISM).

From the results of the above described sequencing, it was found that gene mutations shown in Table 1 were present.

TABLE 1
Detection of gene mutation (No. 1)

| Sample | Mutation site | | | | |
|---|---|---|---|---|---|
| (Cell | 1* (1) | 2* (2-2) | 2* (2-5) | 3* (3-2) | 4* (4) |
| line) | (−1431) | (−1027) | (−912) | (−472) | (−108) |
| KB | — | ◯ | ◯ | ◯ | — |
| SCC-9 | — | — | — | — | ◯ |
| SAS | ◯ | ◯ | — | ◯ | — |
| HSC-2 | ◯ | ◯ | ◯ | — | — |
| HSC-3 | — | ◯ | — | — | — |
| HSC-4 | ◯ | — | ◯ | ◯ | ◯ |
| Ca9-22 | — | — | — | — | — |
| OSC-19 | ◯ | ◯ | ◯ | ◯ | — |
| OSC-20 | ◯ | ◯ | ◯ | ◯ | ◯ |

◯: mutation detected
−: no mutation detected
*shows primer sets 1 to 4 used for amplification.

### Example 2

Detection by SSCP Method

DNA was extracted from the above described 9 types of cultured cell lines. The extraction of DNA from cultured cells was carried out using Sepa Gene (Sanko Jyunyaku). Using AmpliTaq Gold Master Mix (ABI PRISM), the extracted DNA was amplified with FITC-labeled primers (the same 4 types of primers as those used in the direct sequencing, which were labeled with FITC), using Thermalcycler (TaKaRa). A part of the thus obtained PCR product was subjected to electrophoresis in which NuSieve 3:1 agarose (1.5% Agarose gel) (BMA) was used, so as to confirm that it was a single band. Thereafter, the PCR product was subjected to heat denaturation at 95° C. for 5 minutes followed by quenching. The obtained product was subjected to electrophoresis of using gel prepared from 6% Long Ranger Gel Solution (BMA). Then, using FMBIO Read Image (FMBIO Analysis V8.0), the presence or absence of a gene mutation was analyzed from the difference in the position of a single stranded DNA band.

As a result, the difference in the position of the bands was observed and it was therefore found that gene mutations shown in Table 1 were present.

### Example 3

Detection of a Mutation in Human Gene by Direct Sequencing

Genes were isolated from the mucous membrane inside the cheek of each of 61 subjects who agreed on the sampling and test of genes, and the collected genes were then sequenced.

The same operations as in Example 1 were carried out with only exception that, instead of the cultured cells, cells obtained by scrubbing the mucous membrane inside the human cheek with a cotton swab and isolating them with QIAamp DNA Mini Kit (QIAGEN) were used.

From the results of the above sequencing, it was found that gene mutations shown in the following Table 2 were present.

TABLE 2
Detection of gene mutation (No. 2)

| | Mutation site | | | | |
|---|---|---|---|---|---|
| | 2* (2-2) | 2* (2-5) | 3* (3-2) | Number of | Frequency |
| | (−1027) | (−912) | (−472) | detection | (%) |
| Mutation | — | — | — | 0 | 0 |
| pattern 1 | | | | | |
| Mutation | — | — | ◯ | 1 | 1.64 |
| pattern 2 | | | | | |
| Mutation | — | ◯ | ◯ | 5 | 8.20 |
| pattern 3 | | | | | |
| Mutation | — | ◯ | — | 0 | 0 |
| pattern 4 | | | | | |
| Mutation | ◯ | — | — | 0 | 0 |
| pattern 5 | | | | | |
| Mutation | ◯ | ◯ | — | 0 | 0 |
| pattern 6 | | | | | |
| Mutation | ◯ | — | ◯ | 1 | 1.64 |
| pattern 7 | | | | | |
| Mutation | ◯ | ◯ | ◯ | 54 | 88.52 |
| pattern 8 | | | | | |
| Total number | | | | 61 | 100 |

◯: Mutation detected
−: No mutation detected
*shows primer sets 2 and 3 used for amplification

### Example 4

Detection of a Mutation in Human Gene by Direct Sequencing

Genes were isolated from the mucous membrane inside the cheek of each of 55 subjects who agreed on the sampling and test of genes, and the collected genes were then sequenced.

The same operations as in Example 1 were carried out with only exception that, instead of the cultured cells, cells obtained by scrubbing the mucous membrane inside the human cheek with a cotton swab and isolating them with QIAamp DNA Mini Kit (QIAGEN) were used.

As a result, there were found several sites at which the corresponding signal in the autosequencer has a height by 50 to 90% lower than that at other sites. Each signal height of A, T, G and C base at these sites were examined. The base whose signal height is secondary at a site was assumed to exist at the same site in the nucleotide sequence of genes as the base whose signal height is primary. Such sites, bases and their incidence in 55 samples are shown in Table 3.

### Example 5

Detection of a Mutation in Human Gene by an Allele Specific PCR Method

Genes were isolated from the mucous membrane inside the cheek of each of 55 subjects who agreed on the sampling and test of genes. Detection of a mutation in human gene obtained was carried out by an allele specific PCR method. The details of the method are described in the following reference: Hamajima N., Saito T., Matsuo K., Kozaki K., Takahashi T., Tajima K., “PCR with opposed-directional 2 pair primers for multiplicity gene typing”, Japanese Journal of Cancer Research, Volume 91, 9, pp865-868 (2000).

To collect DNA samples, the same operations as described in Example 1 were carried out with only exception that, instead of the cultured cells, cells obtained by scrubbing the mucous membrane inside the human cheek with a cotton swab and isolating them with QIAamp DNA Mini Kit (QIAGEN) were used.

The thus obtained DNA, 0.005 μg/μl was separately reacted with each primer set for the detection of respective nucleotide base at −1027 , −912 and −874 positions under the conditions for PCR reaction shown in Table 4, in a LightCycler (Roche Diagnostic), using a LightCycler Kit DNA (Roche Diagnostic) and QuantiTect SYBR Green PCR (Roche Diagnostic)

The time-course alterations of the degrees of amplification allowed us to confirm an allele base at −1027, −912 and −874 positions and their incidence. As a result, the presence of mutation patterns as shown in Table 3 was confirmed.

TABLE 3
Results of human allele analysis

| Position | −1035 | | −1027 | | −936 | | −923 | |
|---|---|---|---|---|---|---|---|---|
| Homo | GG | 30 | AA | 0 | GG | 54 | CC | 29 |
| Hetero | GT | 25 | AG | 25 | GA | 1 | CT | 26 |
| Homo | TT | 0 | GG | 30 | AA | 0 | TT | 0 |
| Position | −912 | | −874 | | −539 | | −472 | |
| Homo | TT | 2 | GG | 25 | CC | 54 | AA | 0 |
| Hetero | TC | 26 | GA | 29 | CT | 1 | AG | 0 |
| Homo | CC | 27 | AA | 1 | TT | 0 | GG | 55 |

The values in the Table indicate the incidence among 55 specimens.

TABLE 4
Conditions of Allele specific PCR reaction

| | Number | | | Retention |
|---|---|---|---|---|
| Sequence | Of Cycle | Procedure | | Time |
| 1 | 1 | Denature | 95° C. | 2 minutes |
| 2 | 50 | Denature | 95° C. | 0 second |
| | | annealing | 60° C. | 5 seconds |
| | | extension | 72° C. | 10 seconds |
| 3 | 1 | Cool | 40° C. | 1 minute |

Primer set for detection of −1027 position

5′ GATGGGAAACACTTATGAAGG 3′
5′ GGCCGCCAATTGCTACTCTGGGTTACGGAG 3′
5′ TTGCTACTCTGGGTTACGGAA 3′

Primer set for detection of −912 position

5′ CTTTGGGAGCCAGGGCTTTCT 3′
5′ AGGGAGACCACGTGGAGGCCTTGCAGCCCC 3′
5′ ACGTGGAGGCCTTGCAGCCCT 3′

Primer set for detection of −874 position

5′ CGTCACTCAGGGAATGTCAGC 3′
5′ CTTCTCCACCAAATCCCAAGGGCAGTGACA 3′
5′ CAAATCCCAAGGGCAGTGACG 3′

### Example 6

Luciferase Reporter Assay

The gene having the intrinsic normal base at −1027 or −472 mutation position was prepared from the promoter gene of KB cell having mutations at −1027 or −472 position by replacement using the following primer sets and GeneEditor in vitro Site-Direct Mutagenesis (invitrogen, CA) according to the directions for use.

Primer set for −1027 position

5′ ctactctgggttacggaggaaggacagg 3′
5′ cctgtccttcctccgtaacccagagtag 3′

Primer set for -472 position

5′ gaatgtccgagcaatggatagaatt 3′
5′ aattctatccattgctcggacattc 3′

The gene portion comprising the above-mentioned gene part, 1.2 kbp moiety upstream from the transcription initiation point, was incorporated into a pGL3-Basic Vector (Promega), together with the following luciferase gene construct primer set:

5′ tctcaaactgcccttagatcga 3′
5′ ccaggagctgagtctggggagg 3′

and each of the vectors was separately introduced into KB cells. Subsequently a luciferase activity was measured.

Determination of the luciferase activity was carried out by extracting proteins from the gene-introduced cells and followed by measurements using a luminometer (Mini lumat LB9506, Berthold Technolgies GmbH, Germany). If fluorescence is detected in the luminometer, it can be considered that the promoter region has a transcription promoting activity.

As a result, when the mutated bases at the positions −1027 and −472 were replaced with normal bases, the luciferase activity was found to be recovered in the repaired gene; particularly the base repair at −1027 position was found to bring about a considerable recovery in transcription activity. Thus, the mutation at said position was demonstrated to inhibit strongly the expression of defensin 2 protein.

What is claimed is:

1. A method of collecting data for estimating susceptibility to periodontal disease, wherein the method comprises:
in order to detect the presence of a gene mutation and/or a mutation site existing in the promoter region of a human defensin gene in a sample,
by using a nucleotide sequence being a part of the promoter of the defensin gene and comprising a mutation site, as a nucleotide sequence for a probe,
determining
(i) a hybridization site of hybridization between the defensin gene promoter nucleotide sequence in the sample and said probe, and/or
(ii) an amplification ability in gene amplification where primers comprising the nucleotide sequence of said probe are used;
thereby clarifying the change of the activity of the defensin promoter to regulate the expression of the defensin gene based on the thus detected presence of a gene mutation and/or a mutation site.

2. A method of collecting data for estimating susceptibility to periodontal disease, wherein the method comprises:
in order to detect the presence of a gene mutation and/or a mutation site existing in the promoter region of a human β-defensin 2 gene in a sample,
by using a nucleotide sequence being a part of the promoter of the β-defensin 2 gene and comprising a mutant nucleotide, as a nucleotide sequence for a probe,
determining
(i) a hybridization site of hybridization between the β-defensin 2 gene promoter nucleotide sequence in the sample and said probe, and/or
(ii) an amplification ability in gene amplification where primers comprising the nucleotide sequence of said probe are used;
thereby clarifying the change of the activity of the human β-defensin 2 promoter to regulate the expression of the β-defensin 2 gene based on the thus detected presence of a gene mutation and/or a mutation site.

3. A nucleotide sequence used as a probe to obtain data for estimating susceptibility to periodontal disease, wherein the nucleotide sequence is used to detect a mutant type sequence existing in the promoter region of a human β-defensin 2 gene, and comprises at least 5 nucleotides being each of upstream and downstream from a mutation site, otherwise at-least-10-nucleotide-containing sequences of which 3′ terminus is the nucleotide of a mutation site in the promoter nucleotide sequences.

4. The nucleotide sequence used as a probe according to claim 3, wherein said nucleotide sequence is any sequence selected from:
a DNA nucleotide sequence amplified by primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

wherein G is substituted by C at a site −1431 located upstream of the transcription initiation point of the human β-defensin 2 gene, and/or
a DNA nucleotide sequence amplified by primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

wherein G is substituted by T at a site −1035 (mutation site 2-1), and/or A is substituted by G at a site −1027 (mutation site 2-2), and/or G is substituted by A at a site −936 (mutation site 2-3), and/or C is substituted by T at a site −923 (mutation site 2-4),
and/or a DNA nucleotide sequence amplified by the same primer set as above, wherein T is substituted by C at a site −912 (mutation site 2-5), and/or G is substituted by A at a site −874 (mutation site 2-6), and/or
a DNA nucleotide sequence amplified by primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

wherein C is substituted by T at a site −539 (mutation site 3-1), and/or A is substituted by G at a site −472 (mutation site 3-2), and/or
a DNA nucleotide sequence amplified by primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

wherein T is substituted by C at a site −108 (mutation site 4).

5. The nucleotide sequence used as a probe according to claim 3 , wherein said nucleotide sequence is further modified with markers for detection and/or amplification.

6. A primer which comprises both nucleotide sequences of primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

and is used to amplify DNA derived from a human defensin gene.

7. A primer which comprises both nucleotide sequences of primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

and is used to amplify DNA derived from a human defensin gene.

8. A primer which comprises both nucleotide sequences of primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

and is used to amplify DNA derived from a human defensin gene.

9. A primer which comprises both nucleotide sequences of primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

and is used to amplify DNA derived from a human defensin gene.

10. A primer which has any one of the nucleotide sequences used as probes according to claim 4, and is used to determine an amplification ability in gene amplification.

11. The nucleotide sequence used as a probe according to claim 4, wherein said nucleotide sequence is further modified with markers for detection and /or amplification.

12. A kit used to estimate susceptibility to periodontal disease wherein the kit comprises at least one type of a probe comprising a nucleotide sequence to detect a mutant type sequence existing in the promoter region of a human defensin 2 gene, and optionally comprising a primer such as at least 0.5 nucleotides being each of upstream and downstream from a mutation site and otherwise at-least-10-nucleotide-containing sequences of which 3′ terminus is the nucleotide of a mutation site in the promoter nucleotide sequences, so as to detect a mutant gene existing in the promoter region of a human defensin gene.

13. The kit according to claim 12, wherein the nucleotide sequence used as a probe is at least one sequence selected from:
a DNA nucleotide sequence amplified by primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

wherein G is substituted by C at a site −1431 located upstream of the transcription initiation point of the human β-defensin 2 gene, and/or
a DNA nucleotide sequence amplified by primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

wherein G is substituted by T at a site −1035 (mutation site 2-1), and/or A is substituted by G at a site-1027 (mutation site 2-2), and/or G is substituted by A at a site −936 (mutation site 2-3), and/or C is substituted by T at a site −923 (mutation site 2-4), and/or
a DNA nucleotide sequence amplified by the same primer set as above, wherein T is substituted by C at a site −912 (mutation site 2-5), and/or G is substituted by A at a site −874 (mutation site 2-6), and/or
a DNA nucleotide sequence amplified by primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

wherein C is substituted by T at a site −539 (mutation site 3-1), and/or A is substituted by G at a site −472 (mutation site 3-2), and/or
a DNA nucleotide sequence amplified by primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

wherein T is substituted by C at a site −108 (mutation site 4).

14. The kit according to claim 12, further comprising at least one primer selected from:
a primer which comprises both nucleotide sequences of primer set 1:

| | |
|---|---|
| 5′ ATAGGCGTAAGCCATCATCCC 3′ | (SEQ ID NO:1) |
| 5′ CATCCTGGTTCCTCCCTCTTT 3′ | (SEQ ID NO:2) |

and is used to amplify a DNA derived from a human defensin gene;
a primer which comprises both nucleotide sequences of primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

and is used to amplify a DNA derived from a human defensin gene;
a primer which comprises both nucleotide sequences of primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

and is used to amplify a DNA derived from a human defensin gene;
a primer which comprises both nucleotide sequences of primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

and is used to amplify a DNA derived from a human defensin gene.

15. A DNA chip wherein the DNA chip comprises at least one type of a probe comprising a nucleotide sequence to detect a mutant type sequence existing in the promoter region of a human defensin 2 gene, and optionally comprising a primer such as at least 5 nucleotides being each of upstream and downstream from a mutation site and otherwise at-least-10-nucleotide-containing sequences of which 3′ terminus is the nucleotide of a mutation site in the promoter nucleotide sequences, so as to detect a mutant gene existing in the promoter region of a human defensin gene.

16. The DNA chip according to claim 15, wherein the nucleotide sequence used as a probe is at least one sequence selected from:
a DNA nucleotide sequence amplified by primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

wherein G is substituted by C at a site −1431 located upstream of the transcription initiation point of the human β-defensin 2 gene, and/or
a DNA nucleotide sequence amplified by primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

wherein G is substituted by T at a site −1035 (mutation site 2-1), and/or A is substituted by G at a site −1027 (mutation site 2-2), and/or G is substituted by A at a site −936(mutation site 2-3), and/or C is substituted by T at a site −923 (mutation site 2-4), and/or
a DNA nucleotide sequence amplified by the same primer set as above, wherein T is substituted by C at a site −912 (mutation site 2-5), and/or G is substituted by A at a site −874 (mutation site 2-6), and/or
a DNA nucleotide sequence amplified by primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

wherein C is substituted by T at a site −539 (mutation site 3-1), and/or A is substituted by G at a site −472 (mutation site 3-2), and/or
a DNA nucleotide sequence amplified by primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

wherein T is substituted by C at a site −108 (mutation site 4).

17. The DNA chip according to claim 15, further comprising at least one primer selected from:
a primer which comprises both nucleotide sequences of primer set 1:

| | |
|---|---|
| 5′ ATAGGCGTAAGCCATCATGCC 3′ | (SEQ ID NO:1) |
| 5′ CATCCTGGTTCCTCCCTCTTT 3′ | (SEQ ID NO:2) |

and is used to amplify a DNA derived from a human defensin gene;
a primer which comprises both nucleotide sequences of primer set 2:

| | |
|---|---|
| 5′ TGTTTCTCAAACTGCCCTTAG 3′ | (SEQ ID NO:3) |
| 5′ ATGGGATTGTGACTACATGTG 3′ | (SEQ ID NO:4) |

and is used to amplify a DNA derived from a human defensin gene;
a primer which comprises both nucleotide sequences of primer set 3:

| | |
|---|---|
| 5′ TCCGGACCCACTTGAGACTCC 3′ | (SEQ ID NO:5) |
| 5′ GAAAATTCCTCCTATCTTGCA 3′ | (SEQ ID NO:6) |

and is used to amplify a DNA derived from a human defensin gene;
a primer which comprises both nucleotide sequences of primer set 4:

| | |
|---|---|
| 5′ ACTCCATTCACACACTGGGTT 3′ | (SEQ ID NO:7) |
| 5′ AACGAGAAGAGGAGATACAAG 3′ | (SEQ ID NO:8) |

and is used to amplify a DNA derived from a human defensin gene.

18. A method for estimating susceptibility to periodontal disease on the basis of nucleotide sequences revealed by a human allele analysis, using the allele specific PCR method.


SEQUENCE LISTING (24 sequences)

SEQ ID NO: 1 - DNA, 21 bp, human
ataggcgtaagccatcatgcc

SEQ ID NO: 2 - DNA, 21 bp, human
catcctggttcctccctcttt

SEQ ID NO: 3 - DNA, 21 bp, human
tgtttctcaaactgcccttag

SEQ ID NO: 4 - DNA, 21 bp, human
atgggattgtgactacatgtg

SEQ ID NO: 5 - DNA, 21 bp, human
tccggacccacttgagactcc

SEQ ID NO: 6 - DNA, 21 bp, human
gaaaattcctcctatcttgca

SEQ ID NO: 7 - DNA, 21 bp, human
actccattcacacactgggtt

SEQ ID NO: 8 - DNA, 21 bp, human
aacgagaagaggagatacaag

SEQ ID NO: 9 - DNA, 21 bp, human
gatgggaaacacttatgaagg

SEQ ID NO: 10 - DNA, 30 bp, human
ggccgccaattgctactctgggttacggag

SEQ ID NO: 11 - DNA, 21 bp, human
ttgctactctgggttacggaa

SEQ ID NO: 12 - DNA, 21 bp, human
ctttgggagccagggctttct

SEQ ID NO: 13 - DNA, 30 bp, human
agggagaccacgtggaggccttgcagcccc

SEQ ID NO: 14 - DNA, 21 bp, human
acgtggaggccttgcagccct

SEQ ID NO: 15 - DNA, 21 bp, human
cgtcactcagggaatgtcagc

SEQ ID NO: 16 - DNA, 30 bp, human
cttctccaccaaatcccaagggcagtgaca

SEQ ID NO: 17 - DNA, 21 bp, human
caaatcccaagggcagtgacg

SEQ ID NO: 18 - DNA, 28 bp, human
ctactctgggttacggaggaaggacagg

SEQ ID NO: 19 - DNA, 28 bp, human
cctgtccttcctccgtaacccagagtag

SEQ ID NO: 20 - DNA, 25 bp, human
gaatgtccgagcaatggatagaatt

SEQ ID NO: 21 - DNA, 25 bp, human
aattctatccattgctcggacattc

SEQ ID NO: 22 - DNA, 22 bp, human
tctcaaactgcccttagatcga

SEQ ID NO: 23 - DNA, 22 bp, human
ccaggagctgagtctggggagg

SEQ ID NO: 24 - DNA, 4799 bp, human
  beta-defensin 2
gaattcacatttctcaccttttgatgtattaagaaagtatggagaaatatatcctctatcaaattttcatgccttcaataatttctaattcatcagtcagtgtttttccatcctttactgtgatgatgccctttcttccaaactttttcattgcatcagagatgatgttaccaatttctttgtctccatttgcagaaattgtagcaacctgtgcaatttcttcaggtttggtcacaggtttagactgctttttaagttcagcaattacagcatcaacagctaacatcacacctctcttgatttccactggattagcacctttgctaaccttctggaaggcttatttggaaatagagcataccagtacagcagcagtgatagtgccatcccccagtctctccatttgtgttattggcaacatcttggacaagtttagctccaatgcttttatatttatcctttaagtcaattgactttgcatcagtcacaccatcttttgttactttgggacttccccagctatgttcaataattactgttcttccctttggccccattgtaatggctacagcatcgacaaaaagtctacactttgaagcattaaggctcagacatcagcaccaaattttacatctttaccatcacttcaagtgaggtgaggagccagtagcctggacactggtctcatctggtgaaagactgtgggtaatggaagcatttctgtggggtggtggcaggacatgtgcatggtgaggcaggtcatcagcagcaagtgagagctgcctcttactttctaaaggtgacatagcaagtatacaaaaaaaaataaaatattaatttaggcagagcacataaaggctttatttcatattccatttctctgtatgctttcttcaccaggaagaaatagttttagtgtcaggaatgaatgagtctgcccctcaattccagcctgctcagcacacaaggaaacaaagccctgacaatcagagtgactccctggtgactaagctccagtcctggatgcatatttgtttagcagttctgacagcatctgacccagccctctctttgcataccccaccagaaccttctttttttttttttttctttgagactgagtcttgctctgtcggaagcgattcccgtgcctcagcctcccaaatacctggaattataggcgtaagccatcatgcctggctaatttttgtatttttcatggagatggggttttgccatgttggtcaaattggtctcacactcctgacctcatgtgatccacctgcctcagcctcccaaagtgctgggatgacaggtgtaagccaccatgctaggctcagaaatttccttttataaaaatgtcattaaggatcttggctgcacaatatcgttaccagcttcctttaaatccacctctggcctgccaggaatcagggttcttcagaacctgacattttaaatgaagaggtcaggcaggtcatgaggaaagcctcattgtccccatgtctctgtcactgctgcacccctgagacatcacagacatggacactggggcctgcttgtttctcaaactgcccttagatcgaaagagggaggaaccaggatgaatgccactcattttcccaagaaaggccctctcctgagtgcccgggatggggctctgtccattgcctggggccgccaattgctactctgggttacggaagaaggacagggtcctgagagacaccagagacctcacacagccctgaaaacatggggctccttcataagtgtttcccatcaccaacagggagaccacgtggaggccttgcagccctactcggtgcttctccaccaaatcccaagggcagtgacgctgacgtctgtggaaagcagagaaagccctggctcccaaagccctgaagtcctgtggagctgacattccctgagtgacggtgtgaatggaaggaactcaagtgcgggtggtaggccacctcctggcccaggcctgggtgaactctgaggggacacatgtagtcacaatcccatcctcccattctccttctcagaggaaggaagtgggcatccatctgcctcatctctctcccgtggggaagatggggagtttcaggggaactttcacataaatttcaccagctcagatctcctgtgaggatggggcccaccatgctcccggtgctgccagaggccctgagcccctccagggtccctgggtttgagccagccctgtatcatccccaggagctgaatgtccgaacaatggatagaattagatggaaagagctctcaatttggcctgagactgtccccagatactcaggaaaaacaggacgtcgcacagagtgggcagcaggtgagtggcaggttataggtcctgagtttgagtttgttctcacgtgagacagacccagcccctcactccattcacacactgggttttaaatggtgcaagataggaggaattttctggtcccaagagcaggaggaagggattttctggggtttcctgagtccagatttgcataagatctcctgagtgtgcattgttctttgaggaccattctctgactcaccaggtaagtggctgaattctaacctctgtaatgagcattgcacccaataccagttctgaactctacctggtgaccagggaccaggacctttataaggtggaaggcttgatgtcctccccagactcagctcctggtgaagctcccagccatcagccatgagggtcttgtatctcctcttctcgttcctcttcatattcctgatgcctcttccaggtgagatgggccagggaaataggagggttggccaaatggaagaatggcgtagaagttctctgtctcctctcattcccctccacctatctctccctcatccctctctctccttcctctctctgtgtgtcccctccatccttttctcctgcttctctctcttcttccctctctctctttttttctgtctttctttttcctctctccctagagcatgtctttctttctttctctttcctttcttctacccacacttttagactgagtagactgaatgccctatttaattgaaccaagcattgcttccttcaatagaaaaggagtttgagaacccaatggacaactcactcgttcttctaagccaatatgaaggagcccagtagtttgtaaatatcatctcttcactgctttccatgctacaactgctgagactatggttgaaacctgttaggtgactttttaaataaaaggcagaaattttgattttatctaaagaaagtagtatagaatgtcattttctaaatttttatatttaaagagtagatactgcaacctagagaattccagataatcttaaggcccagcctatactgtgagaactactgcagcagacactctgcccccaggacttttctgatcagaggccctgagaacagtccctgccactaggccactgcaggttcacaggacagggacagcccattgaaaccaacttttaaacctggatgcctaaccttcattttctccttgatattatgaaaataaaataaaaaccatgaaaggataaaagagggagagtggaagggaaggatggagaaagggaaaaagaaaatttgagagtaaatcctaaaacaattaatctaatagatatcatcttgtgaaatcctcattttaccaatcttatttatgagtcctgggttttgtgagaacaatggggttctgagaggcaccagagacctcatattttccaaaacctagaacagtataatgaaggaaggagggaaggagggagggagggagggaaggagggaaggagggagggagggagggaaacaaaaagaagaatgaggttgaaaccaggacttagatattagaaacaagccattacaaaatttatttctatggttaattgtggttttcaactgtaagttacttggtgttaatttcctattaaacaatttcagtaagttgcatcttttttatcccatctcagatcaaatacttaacagactaaatgatttgaaaaagcaaaagtttactggcttgtgtgtgttaaaatggaggtatggtggctttgatattatcttcttgtggtggagctgaattcacaagagatcgttgctgagctcctgccagaccccacctggaggccccagtcactcaggagagatcagggtctttcacaatcaggttctacaaaaataaacatcccccaaaccacagcagtgccagtttccatgtcagaaacttagatccaaatgactgactcgcgtctcattatcatgatggaaaagcccaggcttgagaaagaagcccgctgcggatttactcaaggcgatactgacacagggtttgtgtttttccaacatgagttttgagttcttacacgctgtttgctctttttgtgtgttttttccctgttaggtgtttttggtggtataggcgatcctgttacctgccttaagagtggagccatatgtcatccagtcttttgccctagaaggtataaacaaattggcacctgtggtctccctggaacaaaatgctgcaaaaagccatgaggaggccaagaagctgctgtggctgatgcggattcagaaagggctccctcatcagagacgtgcgacatgtaaaccaaattaaactatggtgtccaaagatacgcaatctttatcctagtaattgtggtcattgggtgatgttggtttgggcaggccatctctaatatccttgaaacacctttttctgctctccaggaaggggtcagggctgccacagcggggcttggagtgc